(19) United States
(12) Patent Application Publication
(10) Pub. No.: US 2019/0358220 A1
(43) Pub. Date: Nov. 28, 2019

(54) INTEGRATOR INHIBITORS AND METHODS FOR THEIR USE

(71) Applicant: UNIVERSITY OF MIAMI, Miami, FL (US)

(72) Inventors: Ramin Shiekhattar, Miami, FL (US); Ezra Blumenthal, Miami, FL (US); Jenaro Garcia-Huidobro, Miami, FL (US)

(21) Appl. No.: 16/477,545

(22) PCT Filed: Jan. 12, 2018
(86) PCT No.: PCT/US18/13494

§ 371 Date: Jul. 12, 2019

Related U.S. Application Data
(60) Provisional application No. 62/445,832, filed on Jan. 13, 2017.

(51) Int. Cl. A61K 31/4745

(52) U.S. Cl. CPC A61K 31/4745

(57) ABSTRACT

Disclosed herein are inhibitors of the Integrator complex, and methods for their use in treating or preventing diseases, such as cancer. The inhibitors described herein can include compounds of Formula (I) and pharmaceutically acceptable salts thereof: wherein the substituents are as described.

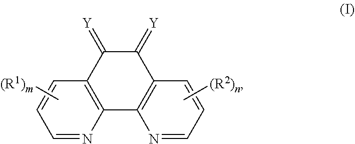

### CROSS REFERENCE TO RELATED APPLICATION

This application claims priority to U.S. Provisional Patent Application No. 62/445,832, filed on Jan. 13, 2017, the disclosure of which is hereby incorporated by reference in its entirety.

### STATEMENT OF GOVERNMENT SUPPORT

This invention was made with government support under grant number 2R01GM078455-06 awarded by the National Institute of General Medical Sciences of the National Institute of Health. The Government has certain rights in the invention.

### BACKGROUND OF THE INVENTION

### Field of the Invention

The present disclosure relates methods of inhibiting the RNA polymerase II Integrator complex to attenuate growth signal response and to reduce the viability of cancer cells.

### Description of Related Technology

The canonical mitogen-activated protein kinase (“MAPK”) or extracellular signal-related kinase (“ERK1/2”) cascade is one of the key signaling pathways that transmits growth signals to the nucleus. See Karin, M. & Hunter, T. Current Biology 5, 747-757 (1995); Gonzalez, F. A. et al. The Journal of Cell Biology 122, 268 1089-1101 (1993); Chen, R. H., Sarnecki, C. & Blenis, J. Molecular and Cellular Biology 12, 915-927 (1992). Following its activation, ERK governs a multitude of transcription factors that regulate expression of genes involved in fundamental cellular processes including, proliferation, differentiation, survival, and motility. See Roux, P. P. & Blenis, J. Microbiology and Molecular Biology Reviews 68, 320-344, (2004). Over 150 substrates of ERK have been identified and notably, about half are localized in the nucleus. See Yoon, S. & Seger, R. Growth factors 24, 21-44, (2006). Perhaps the most studied response following ERK activation is the phosphorylation of transcription factors that promote expression of immediate early genes (“IEGs”). See Foulds, C. E., Nelson, M. L., Blaszczak, A. G. & Graves, B. J. Molecular and Cellular Biology 24, 10954-10964, (2004); Nelson, M. L. et al. Proceedings of the National Academy of Sciences of the United States of America 107, 10026-10031 (2010).

Murphy, L. O., Smith, S., Chen, R. H., Fingar, D. C. & Blenis, J. Nature Cell Biology 4, 556-564 (2002). Despite the identification of many of these substrates, which include the ETS family members ELK1 and ETS1/2, the precise molecular mechanism by which ERK1/2 activates the expression program of IEGs is strikingly unclear.

Integrator, a RNA polymerase II-associated co-activator complex, plays a vital role in the transcriptional response following ERK1/2 signaling. Integrator depletion diminishes ERK1/2-transcriptional responsiveness and cellular growth in human cancers harboring activating mutations in MAPK signaling. Pharmacological inhibition of ERK1/2 abrogates the stimulus-dependent recruitment of Integrator. In particular, the Integrator complex is recruited to the IEGs to coordinate transcriptional initiation and pause release during epidermal growth factor (EGF) stimulation. See Gardini, A. et al. Molecular Cell 56, 128-139 (2014). Integrator is also directed to enhancers where it facilitates transcription of enhancer RNAs and mediates their 3′-end processing. See Lai, F., Gardini, A., Zhang, A. & Shiekhattar, R. Nature 525, 399-403 (2015).

Approximately two-thirds of human cancers, including colon, lung, pancreas, hairy cell leukemia, and skin, have aberrations in the ERK1/2 cascade, largely due to activating mutations in signaling intermediates, such as EGFR, KRAS or BRAF. See Garnett, M. J. & Marais, R. Cancer Cell 6, 313-319, (2004); Dhillon, A. S., Hagan, S., Rath, O. & Kolch, W. Oncogene 26, 3279-3290, (2007); Davies, H. et al. Nature 417, 949-954, (2002); Bryant, K. L., Mancias, J. D., Kimmelman, A. C. & Der, C. J. Trends in Biochemical Sciences 39, 91-100, (2014). This understanding led to the development of targeted inhibitors against kinase components of the MAPK pathway that could be used for cancer therapy. See Santarpia, L., Lippman, S. M. & El-Naggar, A. K. Expert Opinion on Therapeutic Targets 16, 103-119, (2012); Roberts, P. J. & Der, C. J. Oncogene 26, 3291-3310, (2007). However, the rapid emergence of resistance towards these inhibitors has hindered their therapeutic efficacy. See Samatar, A. A. & Poulikakos, P. I. Nature Reviews. Drug Discovery 13, 928-942, (2014).

Thus, there is a need for new therapeutic targets in growth factor signalizing, and methods for arresting or decreasing tumor growth using these targets.

### SUMMARY OF THE INVENTION

Provided herein are methods of inhibiting Integrator in a cell comprising contacting a cell with a compound of Formula (I) or pharmaceutically acceptable salt thereof in an amount effective to inhibit Integrator. In some cases, the Integrator is INTS11.

Also provided are methods of suppressing mitogen-activated protein kinase (MAPK) signaling in a cell comprising contacting the cell with a compound of Formula (I) or pharmaceutically acceptable salt thereof in an amount effective to suppressing MAPK signaling.

In various cases, the contacting is in vivo. In various cases, the contacting can comprise administering to a patient in need thereof. In some cases, the patient suffers from a disease associated with aberrant MAPK signaling in a cell. In various cases, the disease is cancer. In some cases, the cancer is selected from the group consisting of pancreatic, ovarian, prostate, breast, liver, uterine, bladder, lung, esophagus, diffuse large B-cell lymphoma, uveal melanoma, cholangiocarcinoma, stomach, sarcoma, testicular, malignant peripheral nerve sheath tumors, head and neck, mesothelioma, colorectal, cervical, and combinations thereof.

The compound of formula (I) has a structure

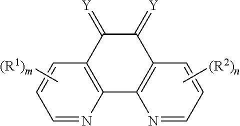

wherein each Y independently is O or S; each R¹ and R² is independently C₁₋₄alkyl or halo; and each of m and n is independently 0, 1, 2, or 3. In various cases, the compound is as a pharmaceutically acceptable salt. In various cases, at least one Y is O. In some cases, each Y is O. In some cases, at least one of m and n is 0. In some cases, each of m and n is 0. In some cases, at least one of m and n is 1, 2, or 3. In some cases, n is 1. In some cases, m is 1. In some cases, n is 2. In some cases, m is 2. In various cases, at least one of R¹ and R² is methyl, ethyl or propyl. In some cases, at least one of R¹ and R² is fluoro, chloro, or bromo.

### BRIEF DESCRIPTION OF THE DRAWINGS

FIG. 1 depicts a model of Integrator in the MAPK signaling pathway. Following Epidermal Growth Factor stimulation, ERK1/2 is phosphorylated and activated via the canonical MAPK signaling pathway. Phosphorylated ERK1/2 trans-locates to the nucleus and recruits Integrator to activate ERK1/2-responsive genes.

FIG. 2 depicts immunoblots of whole cell lysates showing the inhibition of ERK1/2 kinase and its downstream target (FIG. 2A) and FLAG affinity purified proteins detected with antibody against phosphor-serine and threonine (FIG. 2B). In particular, HeLa cells with exogenous expression of FLAG-INTS11 were maintained in serum-depleted medium for two days, and then treated with MEK inhibitor for 3 hours and stimulated with EGF for 20 minutes.

FIG. 3 demonstrates that Integrator is required for the EGF-induced MAPK/ERK1/2 pathway. FIG. 3A depicts a heat map that represents the fold induction of 106 EGF-induced genes in HeLa cells following treatment with Vehicle, ERK inhibitor (SCH772984) or INTS11 knockdown. Each lane represents the fold ratio of gene expression changes before and after 20 minutes of EGF stimulation. The heat map is ranked by EGF-responsive genes displaying highest fold induction. All genes were induced by at least 2-fold. Z-scores were scaled across rows. FIG. 3B shows that EGF-induced gene expression at EGR1, FOSB and NR4A1 loci were diminished by the presence of ERK inhibitor or shRNA against INTS11, as revealed by deep sequencing of chromatin-associated RNA (ChromRNA-seq). The y-axis represents the read counts normalized to sequencing depth. FIG. 3C depicts box plots that represent significant impairments of activation by ERK inhibition or knockdown INTS11. Average expression level of 106 EGF-induced genes and control genes were measured by fold induction after EGF treatment. (Two-sided t-test, ***P<0.001 for all comparisons). FIG. 3D shows that ERK1/2 inhibition or INTS11 knockdown restrains the activation of EGF-responsive enhancers adjacent to EGR1 and CCNL1 gene loci. FIG. 3E depicts box plots that indicate similar inhibition of 75 EGF-induced eRNAs by ERK inhibitor or INTS11 knockdown. (Two-sided t-test, ***P<0.001 for corresponding comparisons). FIG. 3F shows that the activation of enhancers and super-enhancers were repressed by ERK inhibition (green) or INTS11 knockdown (red).

FIG. 4. shows that ERK1/2 inhibition attenuates EGF transcriptional responsiveness in HeLa calls. FIG. 4A is an immunoblot of HeLa cells that were transduced with doxycycline-inducible shRNAs targeting INTS11 and treated with ERK inhibitor (SCH772984, 1 μM) for 3 hours. FIG. 4B shows that ChromRNA-seq reveals ERK inhibition or INTS11 knockdown restrain transcriptional activation of Super-Enhancer and protein coding regions of DUSP5 locus.

FIG. 5. shows that inhibition of MAPK/ERK1/2 diminishes EGF-induced Integrator recruitment. FIG. 5A demonstrates that the presence of ERK inhibitor (SCH772984) affects the dynamic of INTS11 and RNAPII recruitments at EGR1, FOSB and NR4A1 loci. Diagrams of EGR1, FOSB and NR4A1 genomic regions are indicated at the bottom. FIGS. 5B and 5C show the average profiles of INTS11 (top) and RNAPII (bottom) recruitment at 106 EGF-induced genes (FIG. 5B) and 106 control genes (FIG. 5C). ChIP-seq was performed before and after 20 minutes of EGF-induction, with or without the presence of ERK inhibitor. The average analysis was performed using two independent biological replicates. FIG. 5D shows that the RNAPII traveling ratio of EGF-induced genes were measured as the ratio between RNAPII density close to the transcription state site and 2.5 kb downstream of each gene. FIG. 5E shows the average profiles of INTS11 and RNAPII recruitments at 75 EGF-induced enhancers.

FIG. 6 shows that the deficiency of INTS11 impairs MAPK transcriptional responsiveness in cancer cells with KRAS and BRAF activating mutations. FIGS. 6A and 6B show immnoblots of KRAS-mutant lung cancer cell line A549 (FIG. 6A) and BRAF mutant melanoma cell line A375 (FIG. 6B). The cells were treated for 3 hours with RAF inhibitor at 1 μM, MEK inhibitor at 200 nM, ERK inhibitor at 1 μM, and phendione at 5 μM of final concentration, respectively. To knockdown INTS 11, the cells were transfected with siRNA and collected at 72 hours after transfection. The cells were maintained in serum-depleted medium for 2 days before harvesting. FIG. 6C shows heat maps illustrating the expression level of MAPK responsive genes in serum-depleted A375 cells. FIG. 6D depicts box plots showing the expression level of MAPK responsive genes (top) and control genes (bottom) in A375 cells. Two-sided t-test, ***P<0.001 for corresponding comparisons.

FIG. 7 shows that Integrator directs MAPK transcriptional responsiveness in cancers with MAPK activating mutations. FIGs. A, B, and C represent lung adenocarcinoma cells (A549) with KRAS activating mutation. FIGs. D, E, F, represent melanoma cells with V600E BRAF mutation (A375). The heat map represents the activation of EGF responsive genes in A549 cells (FIG. 7A) or A375 cells (FIG. 7D) treated with DMSO, ERK1/2 inhibitor SCH772984, MEK inhibitor PD0325901, or BRAF inhibitor Vemurafenib (left); siRNA against GFP or INTS11 (right). FIGs. B and E show Chromatin RNA-seq analysis of EGF-induced gene expression at EGR1, FOSB and NR4A1 loci were restrained by ERK1/2 inhibition, MEK inhibition, BRAF inhibition or siRNA against INTS11. The box plots represent the fold induction of EGF-responsive genes (FIG. 7C) and gene expression level of MAPK-responsive genes (FIG. 7F) with MAPK inhibitors or siRNA against INTS11. (Two-sided t-test, ***P<0.001 for corresponding comparisons). FIG. 8 shows that phendione inhibits Integrator catalytic activity and MAPK responsiveness. FIG. 8A shows phendione treatment, or INTS11 knockdown inhibit snRNA 3′-end processing. Real-time PCR was performed against long form of RNU11 and RNU12 with three independent biological replicates. FIG. 8B shows that Chromtin RNA-seq reveals the extension of UsnRNAs at RNU11 and RNU12 loci by phendione. FIG. 8C shows a heat map and FIG. 8D shows a box plot indicating that EGF induced gene expression was blocked by phendione. FIG. 8E shows that EGF-induced gene expression at EGR1, NR4A1 and DUSP5 loci were restrained by phendione. FIGS. 8F and 8G shows that phendione impedes EFG responsiveness at enhancers (FIG. 8F) and super enhancers (FIG. 8G).

FIG. 9 shows that phendione treatment causes a robust accumulation of unprocessed transcripts of small nuclear RNAs (snRNAs) in HeLa cells. Quantification of RNU12 extended the transcript level detected by real-time PCR in HeLa cells under the treatment of phendione and its derivatives at a final concentration of 10 μM. INTS 11 knockdown, as described in FIG. 6B. The data is presented as a mean (n=3).

FIG. 10 shows that phendione suppresses cell proliferation in cancer cells resistant to MAPK inhibitors. FIGS. 10A and 10B show that INTS11 knockdown inhibits proliferation of EGFR-mutant H1650 lung adenocarcinoma cells, as demonstrated by reduced EdU (Alexa549) incorporation. FIG. 10C shows that phendione treatment impairs viability of cancer cells bearing activating mutations in MAPK signaling components. Lung cancer cell lines (NCI-H1975, NCI-H2444, NCI-H1650) and melanoma cell lines (SK-MEL28 and A375) were treated with phendione or the indicated inhibitors for 96 hours. Cell viability was determined by Prestoblue and presented as percent of vehicle. Three independent biological replicates were used to calculate the average. FIG. 10D shows that A375 cells were rendered resistant to BRAF inhibition. FIG. 10E shows that phendione reduces viability of BRAF-inhibitor-resistant A375 cells. Two-sided t-test, **P<0.01 for corresponding comparisons.

FIG. 11 shows depletion of INTS11 using RNAi in the lung cancer cell line H1650 and PEO4 ovarian cancer cells. The data presented are the quantification of INTS11 transcript level detected by real-time PCR (n=3, two-sided t-test, ***P<0.0001).

FIG. 12 shows that cisplatin-resistant ovarian cancer cells display sensitivity to phendione treatment. FIGS. 12A and 12B show that INTS11 knockdown inhibits proliferation of PEO4 ovarian adenocarcinoma cells, as demonstrated by reduced EdU incorporation. FIG. 12C shows that phendione treatment impairs viability of cisplatin-resistant (A2780, OVCAR10) and cisplatin-sensitive (PEO1, PEO4) ovarian cancer cells. Indicated cells were treated with phendione or cisplatin for 96 hours. Cell viability was determined by Prestoblue and presented as percent of vehicle. Three independent biological replicates were used to calculate the average. Two-sided t-test, **P<0.01 for corresponding comparisons.

FIG. 13 shows the cell viability of IMR90 human fibroblast and BEAS-2B normal lung ephithelia cells. The cells were treated with chemotherapeutic drug cisplatin, EGFR, and HER2 tyrosine kinase inhibitor afatnib, mutant EFGR (T790M) inhibitor Osimertinib and phendione. The concentrations of 50% growth inhibition (IC₅₀) for each cell line is presented in the table.

### DETAILED DESCRIPTION OF THE INVENTION

Provided herein are methods for inhibiting the RNA polymerase II Integrator complex (“Integrator complex”) which can arrest or decrease tumor growth. Integrator is a downstream node of MAPK signaling in the nucleus. It has been found that knockdown of the catalytic subunit of Integrator, INTS11, attenuates ERK1/2-transcriptional responsiveness and reduces growth of cancer cells harboring activating mutations in the MAPK pathway. Therefore, inhibiting the Integrator complex abolishes MAPK transcriptional responsiveness following EGF stimulation, and advantageously allows the treatment and prevention of cancer without the development of resistance.

### Definitions

As used herein, “alkyl” refers to straight chained and branched saturated hydrocarbon groups containing one to thirty carbon atoms, for example, one to four carbon atoms (e.g., 1, 2, 3, or 4). The term Cₙ means the alkyl group has “n” carbon atoms. For example, C₃ alkyl refers to an alkyl group that has 3 carbon atoms. C₁-C₄ alkyl refers to an alkyl group having a number of carbon atoms encompassing the entire range (i.e., 1 to 4 carbon atoms), as well as all subgroups (e.g., 1-2, 1-3, 2-3, 2-4, 1, 2, 3, and 4 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “halo” refers to a fluoro, chloro, bromo, or iodo group.

As used herein, the term “therapeutically effective amount” means an amount of a compound or combination of therapeutically active compounds (e.g., an inhibitor described herein, or a combination of inhibitors) that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (i.e., non-human animals) and humans. Particular patients are mammals (e.g., humans). The term patient includes males and females.

As used herein, the term “pharmaceutically acceptable” means that the referenced substance, such as a compound of the present invention, or a formulation containing the compound, or a particular excipient, are safe and suitable for administration to a subject or patient. The term “pharmaceutically acceptable carrier” refers to a medium that does not interfere with the effectiveness of the biological activity of the active ingredient(s) and is not toxic to the host to which it is administered.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

As used herein, the term “deregulation of mitogen-activated protein kinase (“MAPK”) pathway refers to an abnormality in the regulatory ability of the MAPK, resulting in activating mutations in MAPK signaling.

As used herein, the term “aberrant mitogen-activated protein kinase (“MAPK”) signaling” refers to a deviation of MAPK signaling from its normal response.

### Mechanism and Mechanistic Effects of Integrator Inhibition

The mechanistic effects of Integrator were studied and are described in detail below. The experimental procedures used for the Integrator studies can be found in the Examples section, below.

In brief, genetic aberrations in components of the MAPK cascade are an underlying cause of many human cancers. Despite advances toward understanding the molecular basis of MAPK signaling in the cytoplasm, knowledge was lacking of how the activation of ERK1/2, the last cytoplasmic component of the pathway, is translated to a rapid and coordinated transcriptional response in the nucleus. While it was known that ERK1/2 phosphorylates a set of transcription factors, predominantly of ETS-related family members, the precise molecular mechanisms leading to transcriptional induction had not been elucidated. Previous studies have implicated the transcriptional co-activators, CBP/p300 or components of the Mediator complex, in MAPK signaling. See Jun, J. H. et al. The Journal of Biological Chemistry 285, 36410-36419 (2010); Janknecht, R. & Nordheim, Biochemical and Biophysical Research Communications 228, 831-837 (1996); Pandey, P. K. et al. Molecular and Cellular Biology 25, 10695-10710 (2005); Galbraith, M. D. et al, Nucleic Acids Research 41, 10241-10253 (2013); and Wang, G. et al, Molecular Cell 17, 683-694 (2005). However, these studies were generally limited to the analysis of a single or a small number of MAPK-responsive genes in a specific cell line. See Jun, J. H. et al. The Journal of Biological Chemistry 285, 36410-36419 (2010) and Galbraith, M. D. et al, Nucleic Acids Research 41, 10241-10253 (2013).

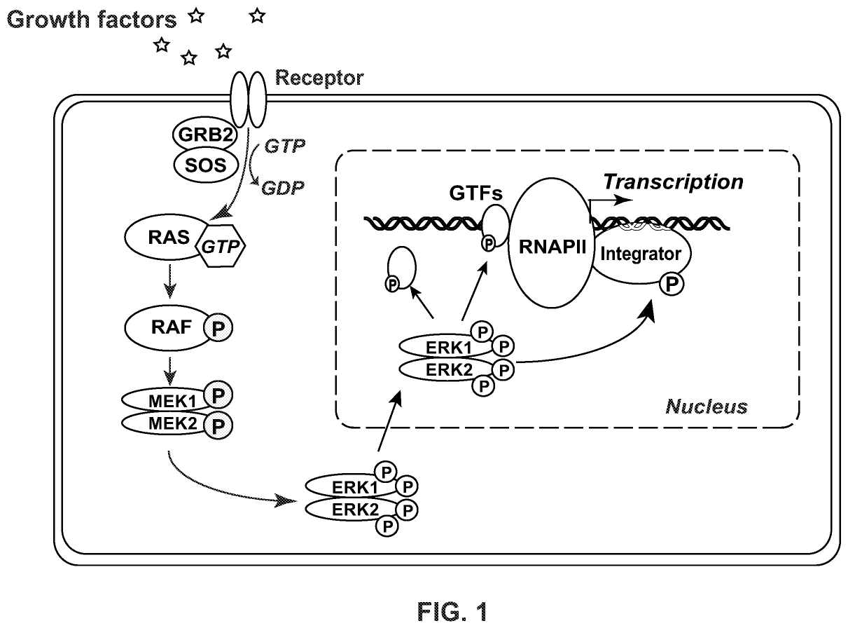

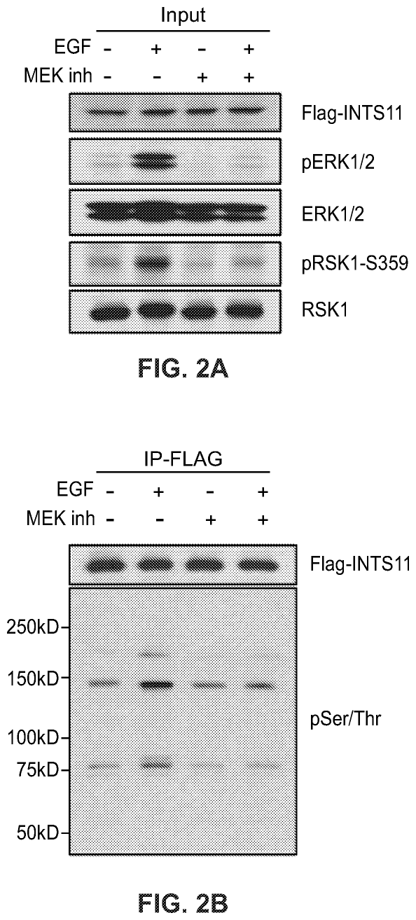

It has now been discovered that Integrator confers the ERK1/2-transcriptional induction to nearly the entire repertoire of MAPK-responsive genes in multiple cancer cell lines, including those with cancer-causing activating mutations in components of MAPK signaling (FIG. 1). In particular, it was found that inhibition of MAPK signaling abrogates the stimulus-dependent recruitment of Integrator, and that ERK1/2 potentially phosphorylates Integrator subunits. See FIG. 2A and FIG. 2B. Without being bound by any particular theory, phosphorylation of a specific transcription factor and/or RNAPII by ERK1/2 could result in increased recruitment of RNAPII and Integrator to MAPK-induced genes. Rowan, B. G., Weigel, N. L. & O'Malley, B. W. The Journal of Biological Chemistry 275, 4475-4483 (2000). Therefore, multiple subunits of Integrator may be targeted by MAPK signaling. See FIGS. 1 and 2. Further, a recent report suggests association of ERK1/2 with chromatin in mouse stem cells. See Tee, W. W., Shen, S. S., Oksuz, O., Narendra, V. & Reinberg, D. Cell 156, 678-690, (2014).28. Therefore, without being bound by any particular theory, it is likely that following activation of the MAPK pathway, ERK1/2 transiently and functionally associates with the transcriptional machinery at MAPK-responsive genes.

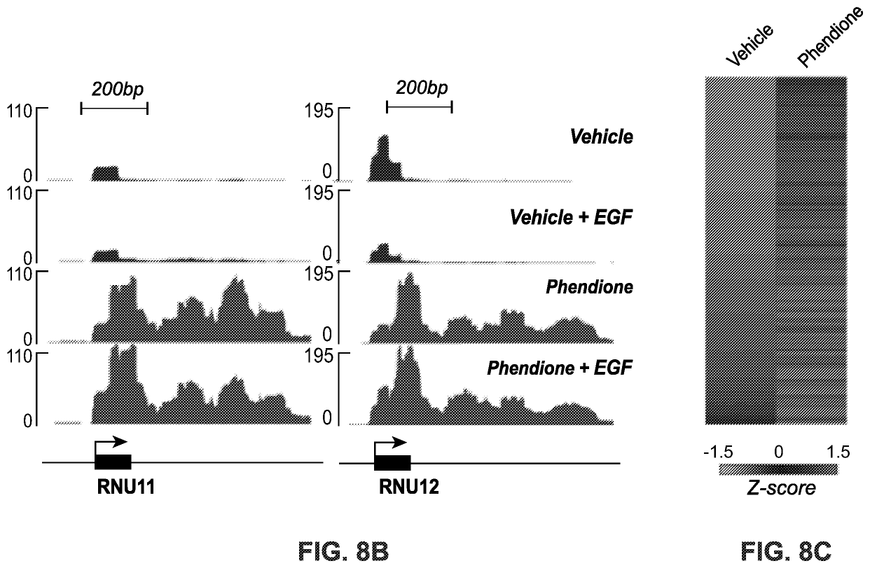

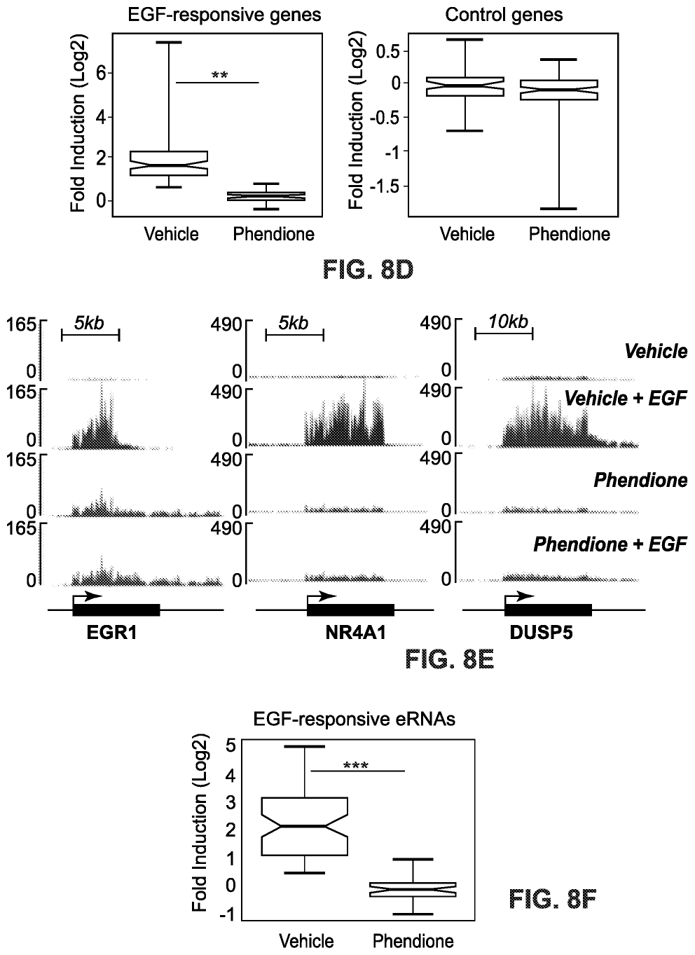

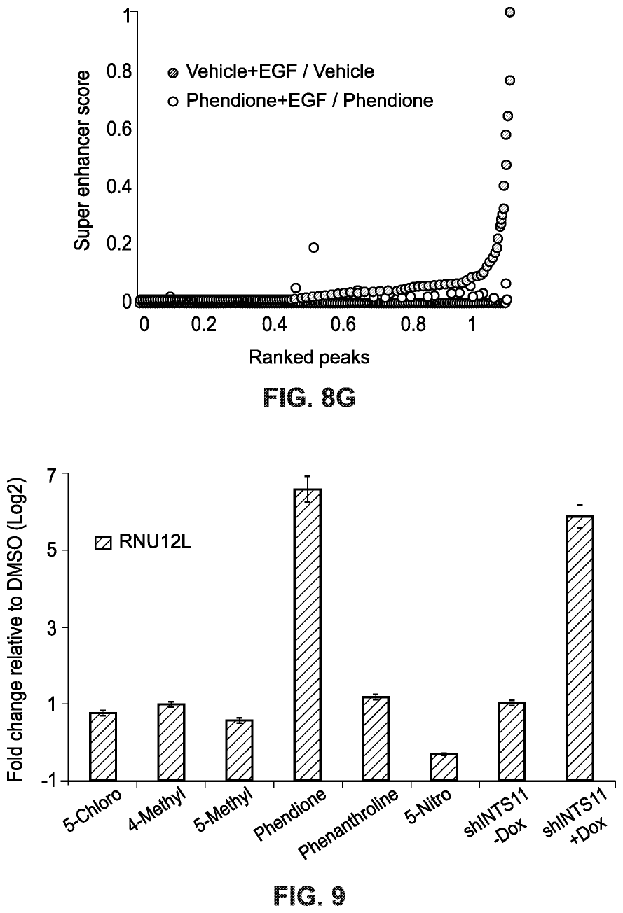

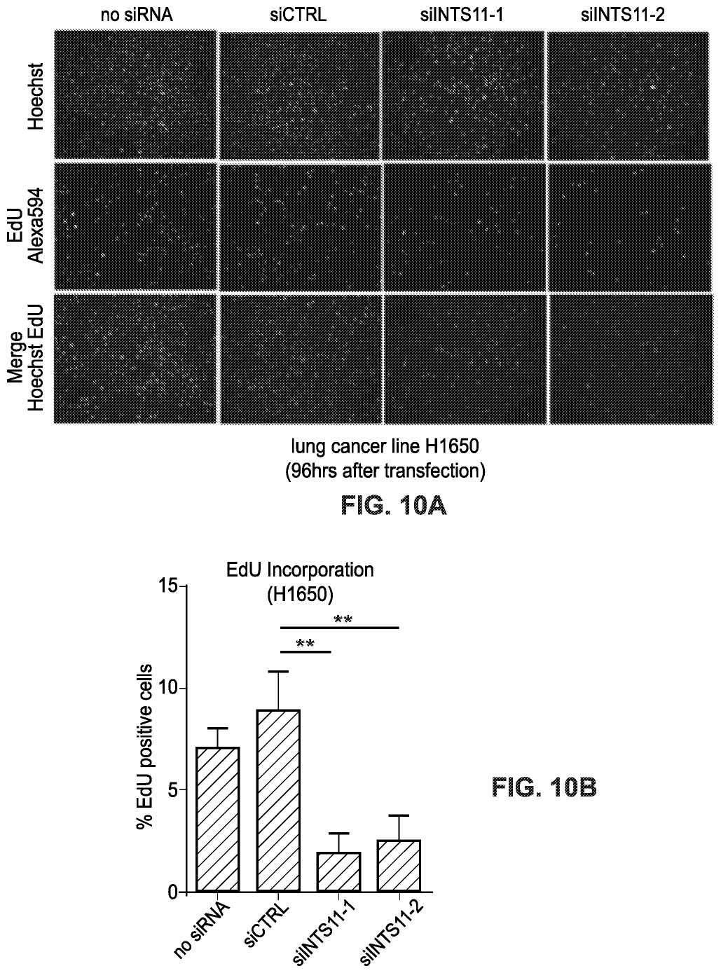

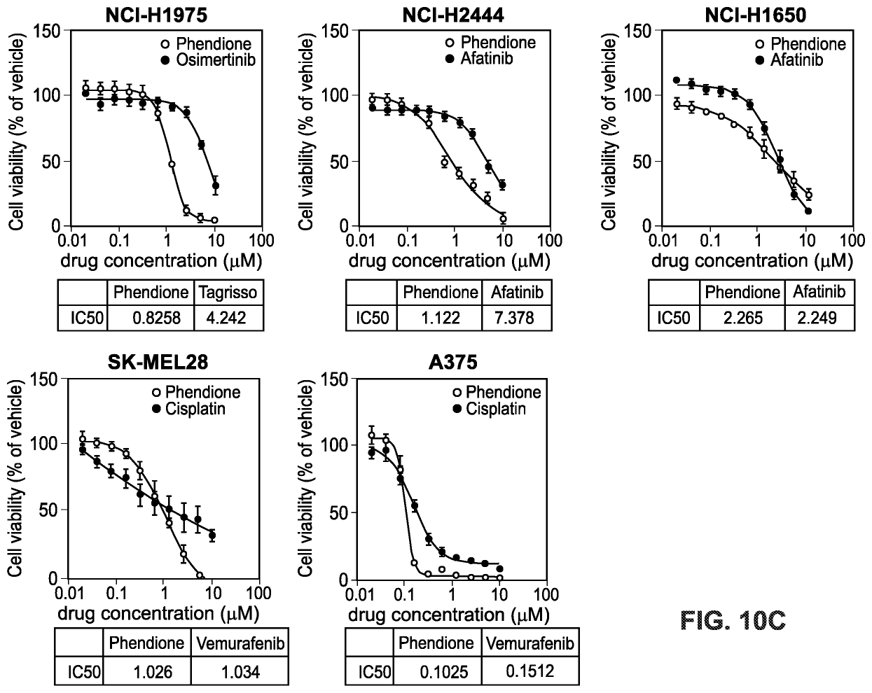

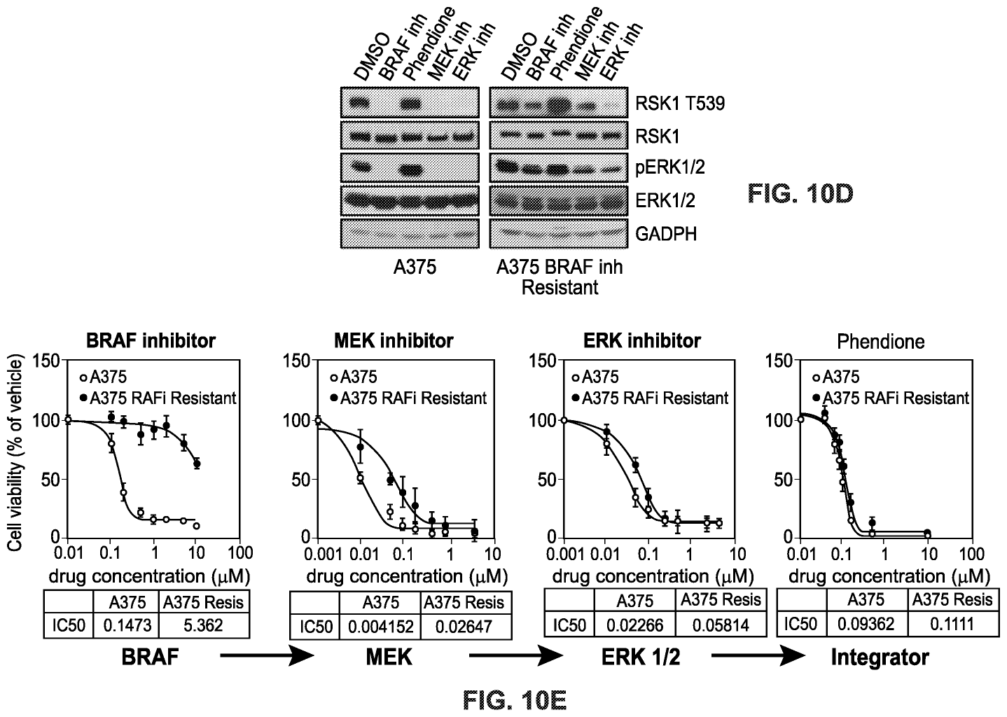

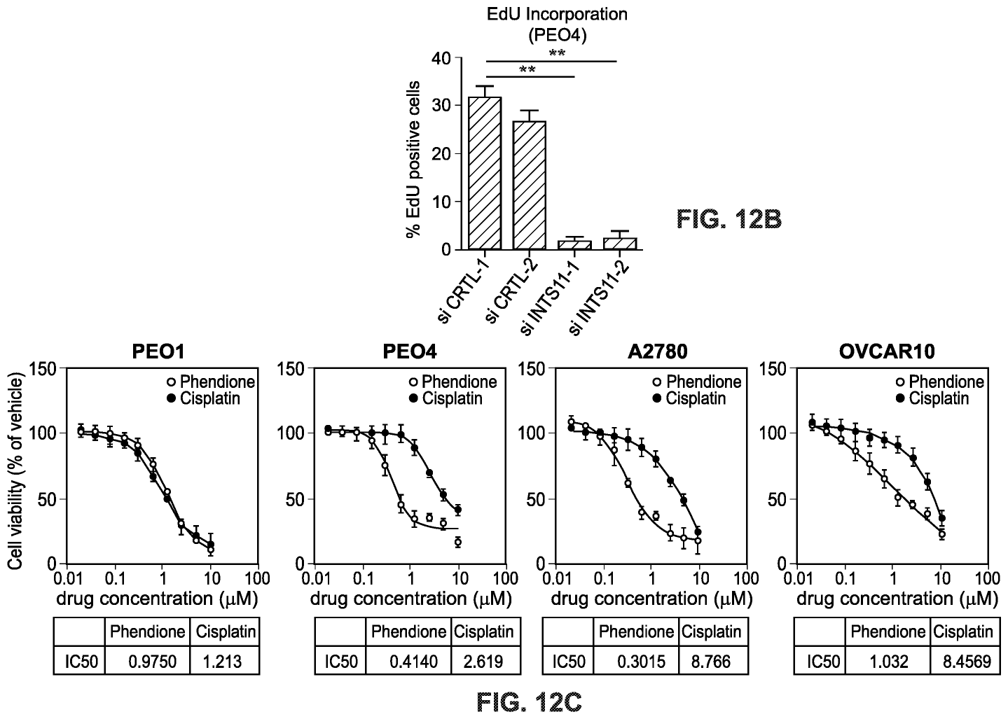

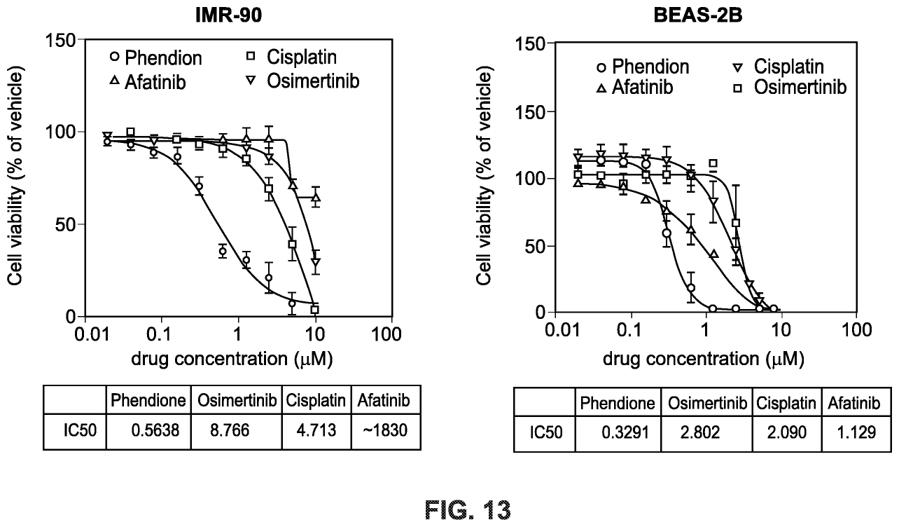

Phendione, but not phenanthroline, is a potent inhibitor of DNA replication through its ability to inhibit INTS11 catalytic activity. See FIG. 9. For example, IMR90 human fibroblasts and BEAS-2B normal lung epithelial cells were found to be sensitive to phendione. See FIG. 13. Phendione also inhibits the related enzyme CPSF73. It also has surprisingly been found that treatment of cancer cells resistant to cisplatin treatment are sensitive to phendione, suggesting a mechanism of action distinct from that of cisplatin. For example, ovarian cancer cells that were resistant to cisplatin treatment were found to be sensitive to phendione. See FIG. 12C. Thus, Integrator can be a potential therapeutic target for cisplatin-resistant cancers. Furthermore, although a melanoma cell line rendered resistant to BRAF inhibitor displayed resistance to MEK and ERK inhibition (FIG. 10E), its response to phendione was not altered.

These results demonstrate that impeding Integrator can be a viable mode of overcoming resistance to MAPK pathway inhibitors, and that phendione can be used to treat cancers with resistance to MAPK inhibitors.

### Integrator is a Key Transcriptional Co-Activator for ERK1/2 Signaling

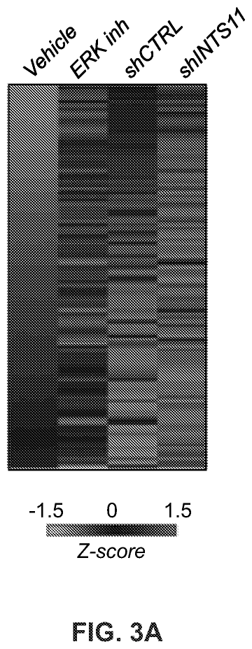

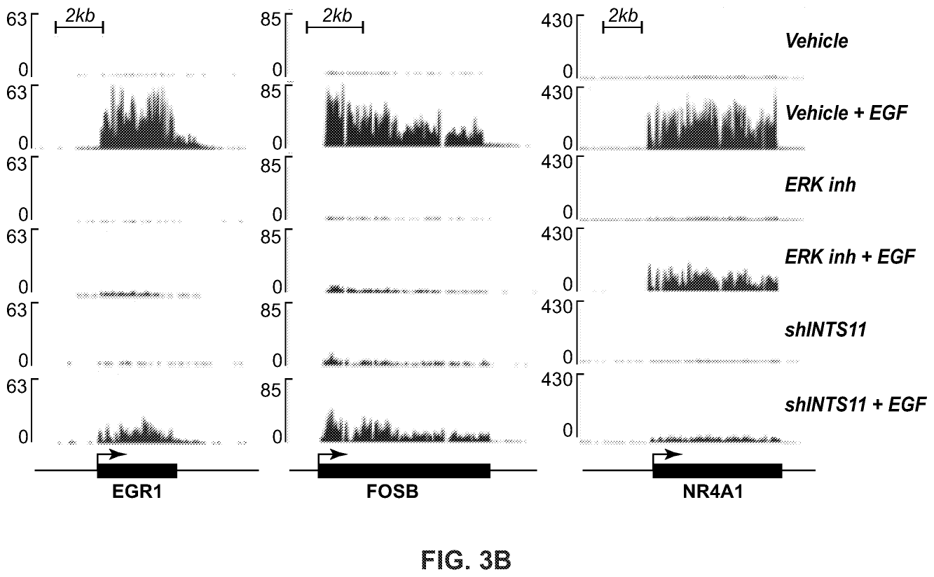

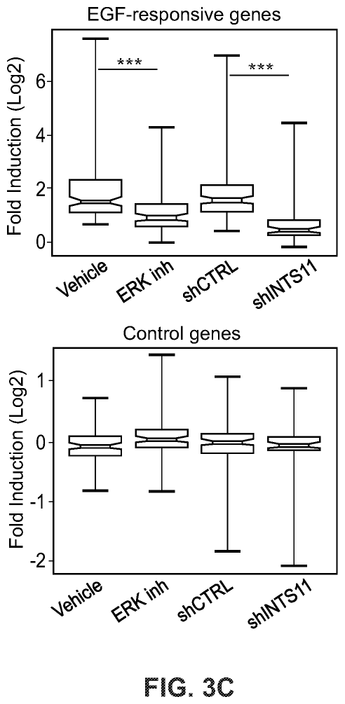

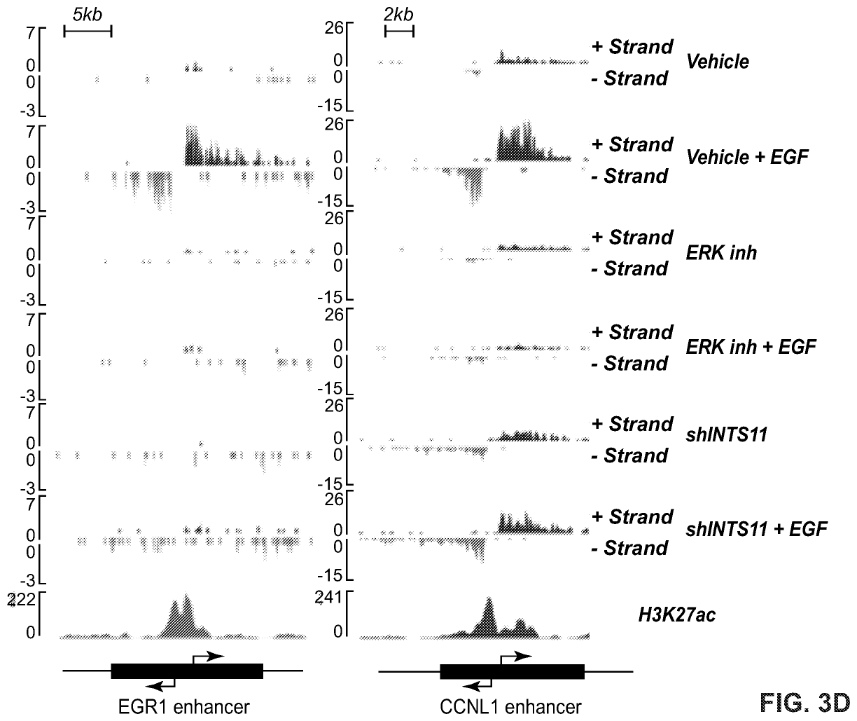

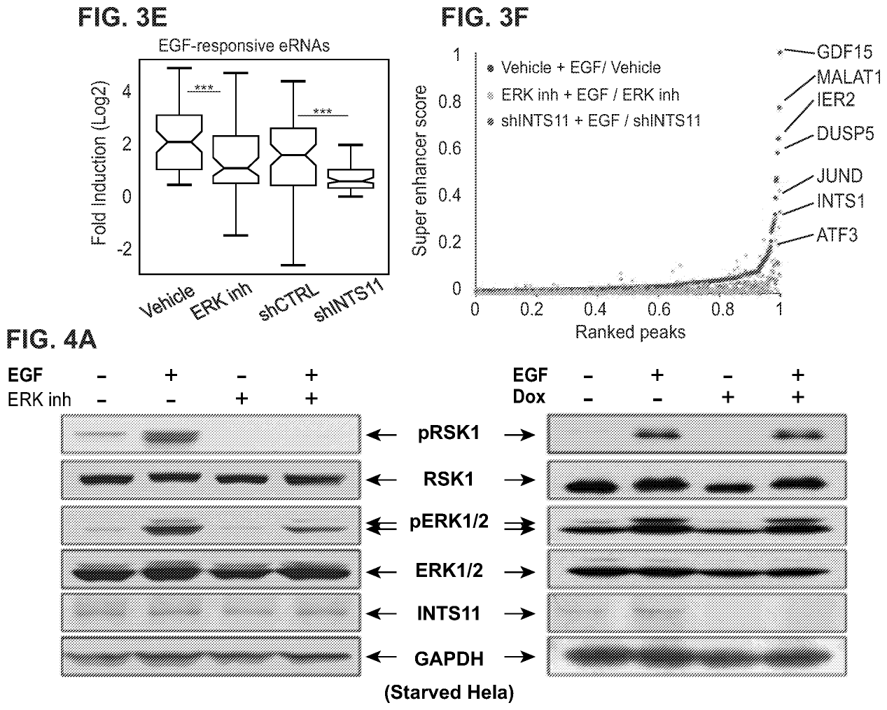

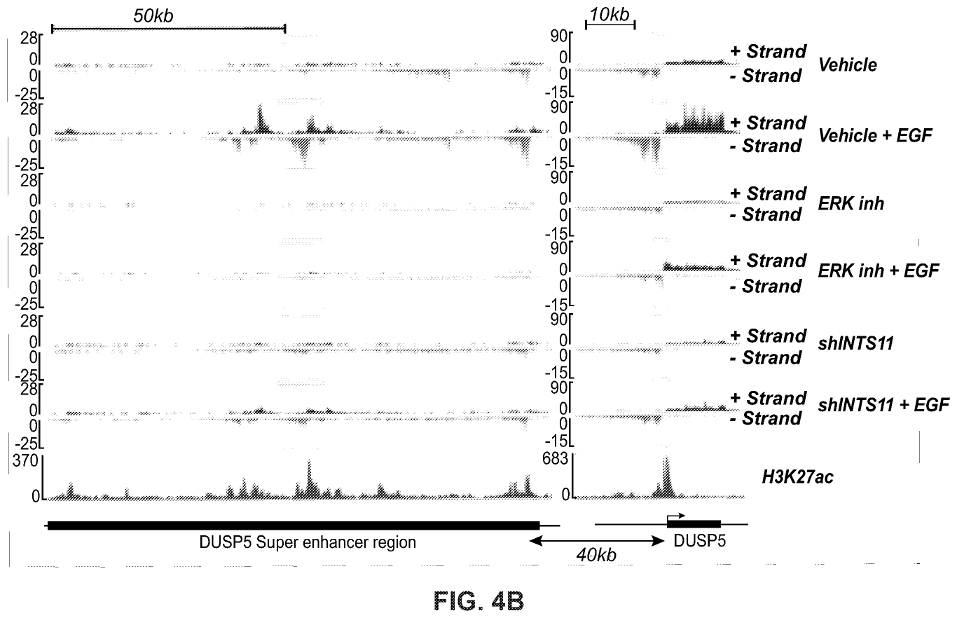

Integrator depletion abrogates EGF transcriptional responsiveness in HeLa cells. See Gardini, A. et al Molecular Cell 56, 128-139 (2014). To dissect the signaling pathway that mediates the EGF transcriptional response of immediate early genes (IEGs), HeLa cells were treated with an ERK1/2 inhibitor (SCH772984) prior to EGF stimulation and analyzed EGF responsive gene expression using Chromatin RNA-sequencing (ChromRNA-seq), which provides for an enriched fraction of nascent RNAs. There were 106 genes that consistently respond (2-fold induction) to EGF stimulation at the 20-minute time point. Inhibition of ERK1/2 resulted in the loss of transcriptional activation of most of EGF-responsive genes (FIGS. 3A and 3B), and abrogation of downstream MAPK-mediated target phosphorylation of RSK1 (FIG. 4A).

The diminished transcriptional response incurred by ERK1/2 inhibition was then compared to that following depletion of INTS11. While Integrator knockdown (INTS11 KD) did not effect ERK1/2 activation (FIG. 4A), it mimicked the pharmacological inhibition of ERK1/2, resulting in the loss of EGF responsiveness (FIGS. 3A, 3B, and 3C; shCTRL examples were similar to Vehicle treatment and are not shown). The effects of ERK1/2 inhibition and Integrator depletion were specific to EGF-responsive genes, as 106 control genes were not affected (FIG. 3C). The enhancer activation was analyzed by measuring the response of EGF-stimulated enhancer RNAs (eRNAs) at enhancers and super-enhancers. ERK1/2 inhibition or INTS11 KD diminished the EGF-induced eRNA induction at enhancers and super-enhancers, similar to that of protein-coding genes (FIGS. 3A-F and FIG. 4B). These results demonstrate that Integrator functions as a critical co-activator of ERK1/2-responsive IEGs within the initial wave of transcriptional activation.

### ERK1/2 Activation Mediates the Recruitment of Integrator to EGF-Responsive Genes

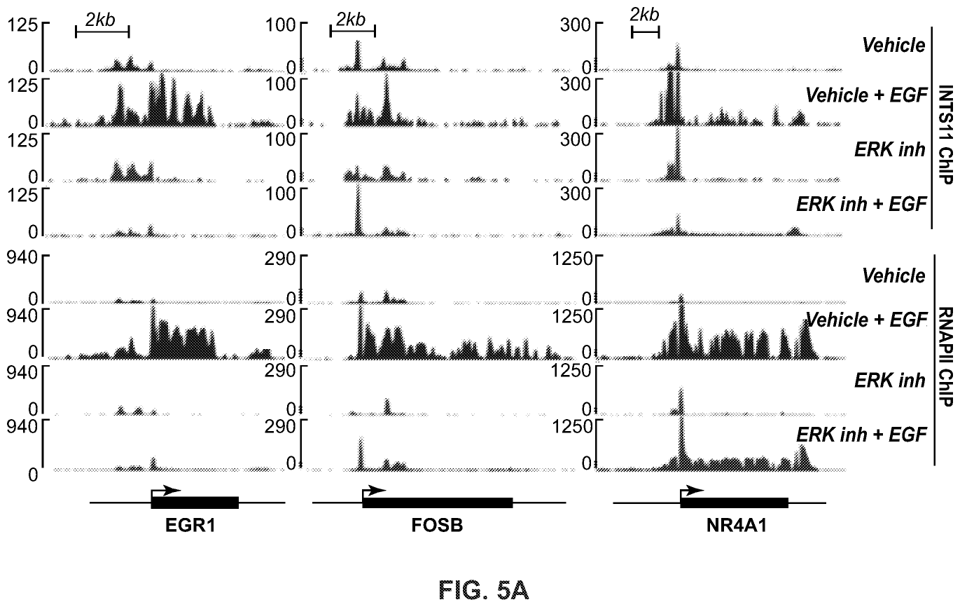

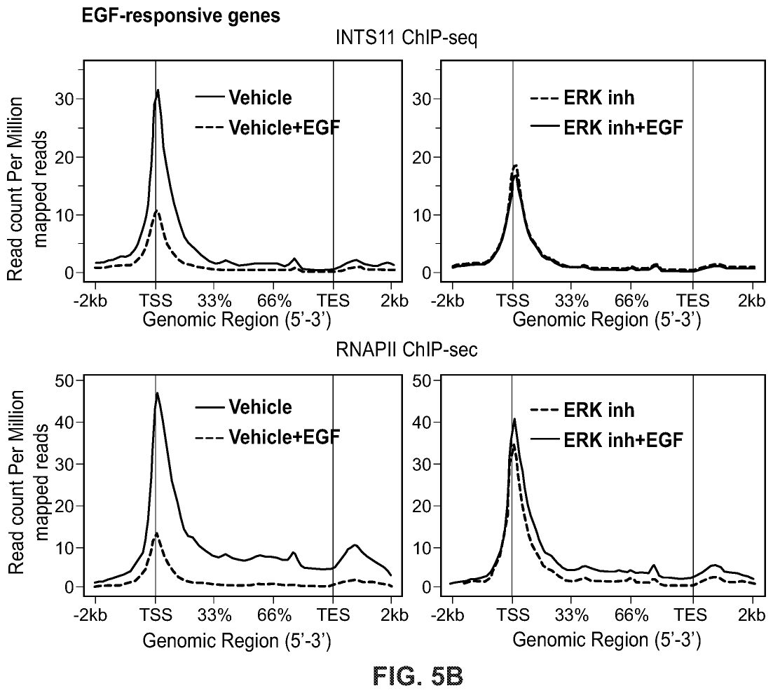

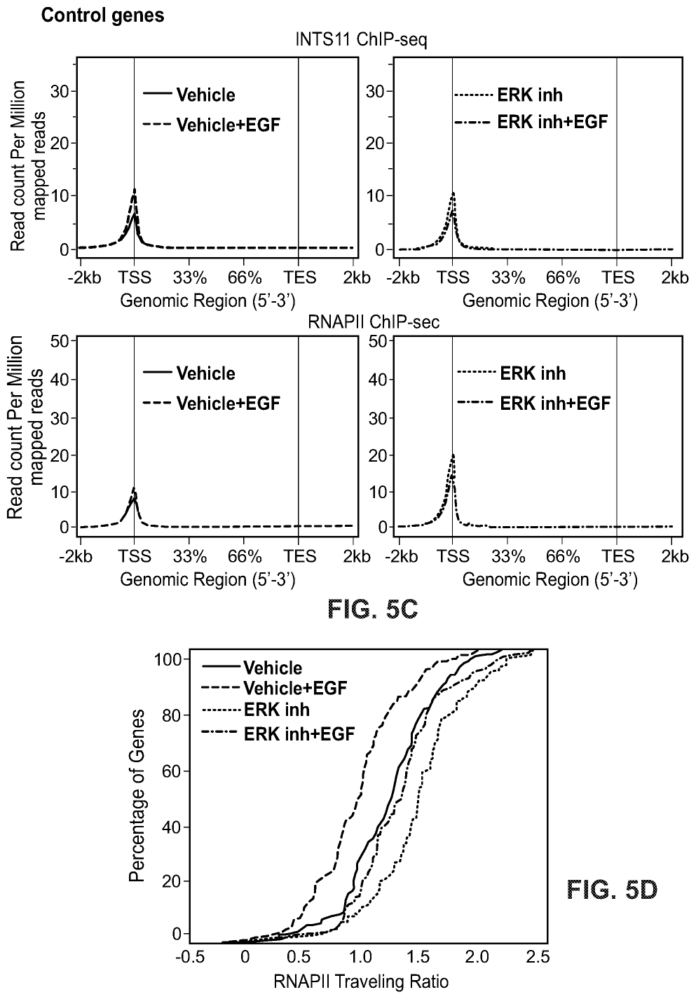

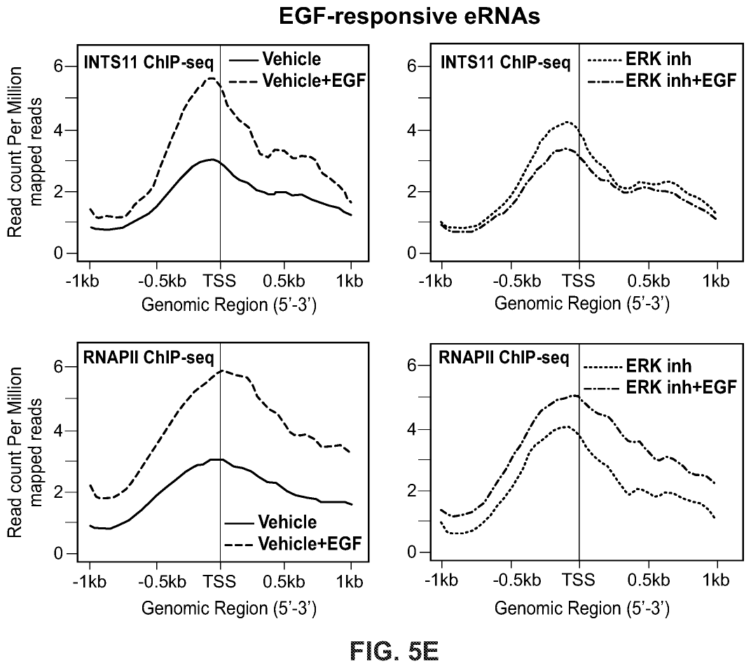

It further was found that ERK1/2-signaling drives Integrator recruitment following EGF-stimulation. ChIP-seq for INTS11 and RNAPII was performed before and after treatment of cells with ERK1/2 inhibitor (SCH772984). Inhibition of ERK1/2 signaling diminished the immediate-early recruitment of Integrator and RNAPII to EGF-responsive IEGs (FIGS. 5A and 5B). This was manifested by decreased occupancy of Integrator and RNAPII at the 5′-end and body of EGF-responsive genes (FIGS. 5A-5C). Analysis of the RNAPII traveling ratio indicated that similar to the effects of INTS11 depletion (Gardini, A. et al. Molecular Cell 56, 128-139 (2014)), ERK1/2 inhibition substantially decreased transcriptional elongation following EGF induction (FIG. 5D). Further, treatment of serum-starved cells with ERK1/2 inhibitor prior to EGF stimulation similarly resulted in increased pausing of RNAPII (FIG. 5D). Moreover, consistent with its effect on eRNA production, ERK1/2 inhibition diminished the recruitment of Integrator and RNAPII to EGF-induced enhancers (FIG. 5E). These results demonstrate that ERK1/2 signaling funnels through the Integrator complex and promotes its recruitment to IEGs. The impaired transcriptional response that follows INTS11 KD indicates that Integrator is a critical downstream component of MAPK signaling in nucleus.

INTS11 KD Diminishes the ERK1/2-Responsiveness in Cancers with Activated MAPK

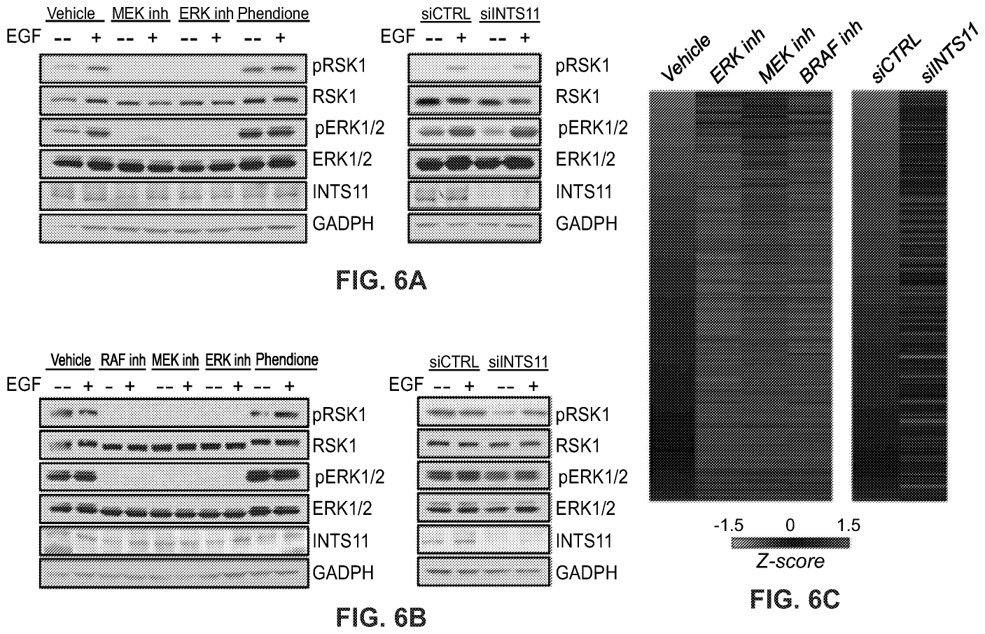

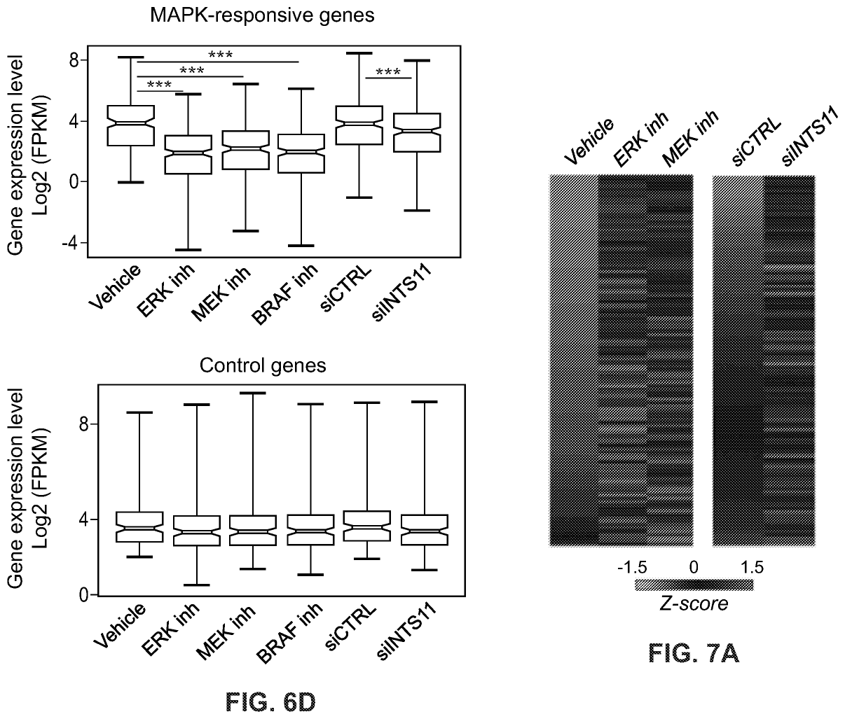

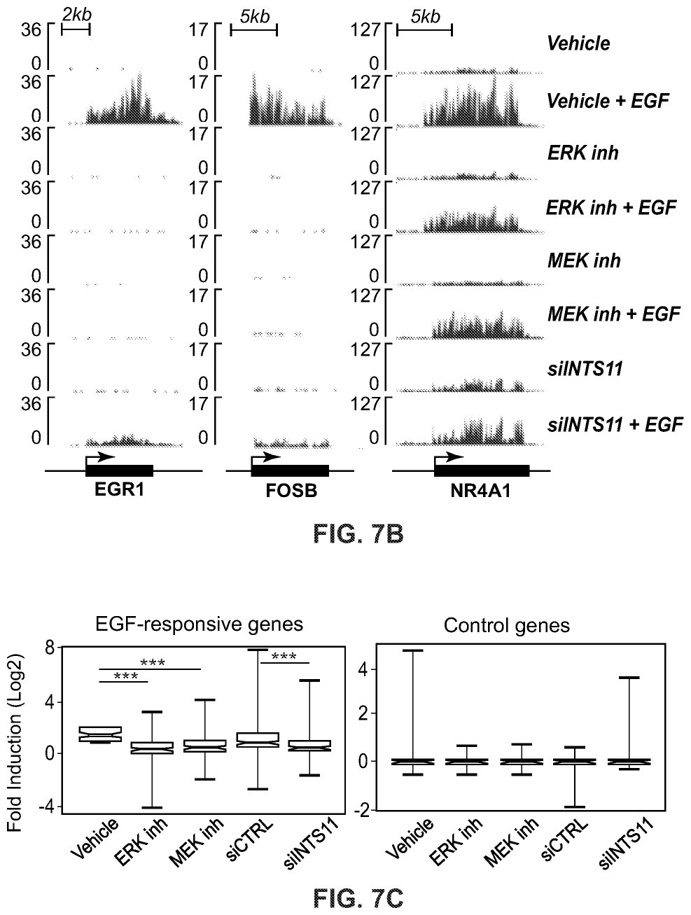

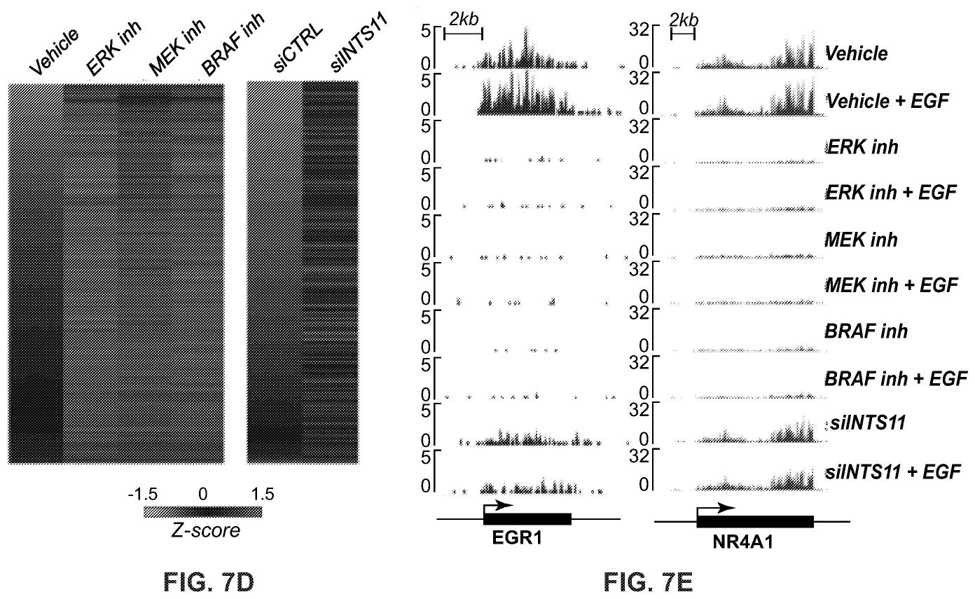

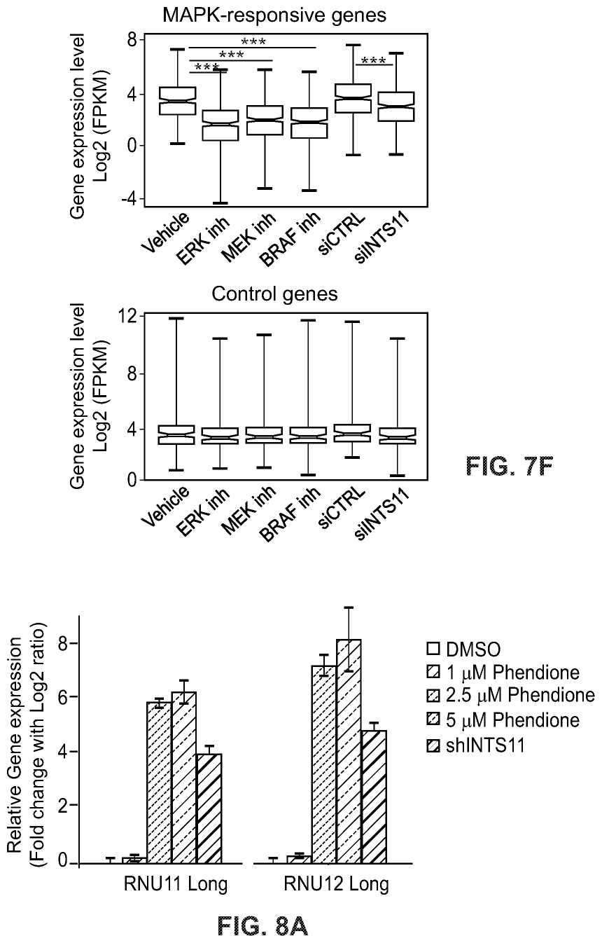

It also was found that INTS11 KD affects the MAPK-mediated responsiveness in cancer cell lines with activating mutations in the MAPK signaling pathway. A549 lung adenocarcinoma cells containing mutations in KRAS (homozygous G12S mutation) were treated with either ERK1/2 or MEK inhibitors (SCH772984 and PD0325901, respectively, prior to stimulation with EGF, similar to the protocols that were used for HeLa cells (FIG. 6A)). Treatment of A549 with either of the MAPK pathway inhibitors specifically diminished the EGF responsiveness of most EGF-responsive genes (112 genes induced by 2 fold) (FIG. 7A-7C). Depletion of Integrator displayed a similar loss of transcriptional induction following EGF stimulation as was observed following treatment with MAPK pathway inhibitors (FIG. 7A-7C).

The analyses was then extended to A375 melanoma cells, which contain an activating V600E mutation in BRAF. A375 cells were treated with inhibitors against mutant BRAF, MEK, and ERK1/2 to arrive at a set of hyper-activated MAPK-responsive genes (319 genes) that diminished their transcription upon treatment with the three inhibitors (FIGS. 7D-7F and FIG. 6B-6D). Interestingly, the V600E mutation in BRAF rendered these cells nearly unresponsive to EGF stimulation (FIG. 7E). Importantly, depletion of INTS11 resulted in a significant cessation of MAPK-responsive transcriptional activation in genes that responded to MAPK pathway inhibitors (FIG. 7D-7F). This was specific, as 319 control genes were unaffected following treatment with MAPK pathway inhibitors or INTS11 KD (FIG. 7F). A375 cells responded similarly to MAPK pathway inhibition or Integrator depletion regardless of EGF stimulation (FIGS. 6C and 6D). Overall, BRAF activated cells displayed a greater inhibition of MAPK-responsive gene expression following treatment with MAPK pathway inhibitors compared to that of Integrator depletion (FIG. 7D). Nevertheless, these results demonstrate that Integrator could be targeted in cancer cells with activating mutations in the MAPK pathway to decrease ERK1/2-mediated transcriptional induction.

### Phendione Inhibits Integrator Catalytic Activity and EGF-Responsiveness

It also was found that phendione inhibits INTS11 enzymatic activity in vivo (FIGS. 8A-8B and FIG. 9). Remarkably, treatment of HeLa cells with phendione (5 μM) specifically abrogated EGF induction of IEGs similar to that observed with either ERK1/2 inhibitor or INTS11 KD (FIG. 8C-8E). Detailed analysis of chromatin RNA-seq indicated that phendione treatment inhibited both related endonucleases INTS11 and CPSF73, as evidenced by extension of reads on the 3′-end of protein-coding genes (FIG. 8E, see for example the 3′-end extension of EGR1). Additionally, phendione treatment diminished eRNA induction at enhancers and super-enhancers (FIGS. 8F and 8G). Taken together, phendione displayed potent inhibitory activity toward Integrator and MAPK-mediated transcriptional induction.

Phendione Inhibits Proliferation of Cancers with Activated MAPK

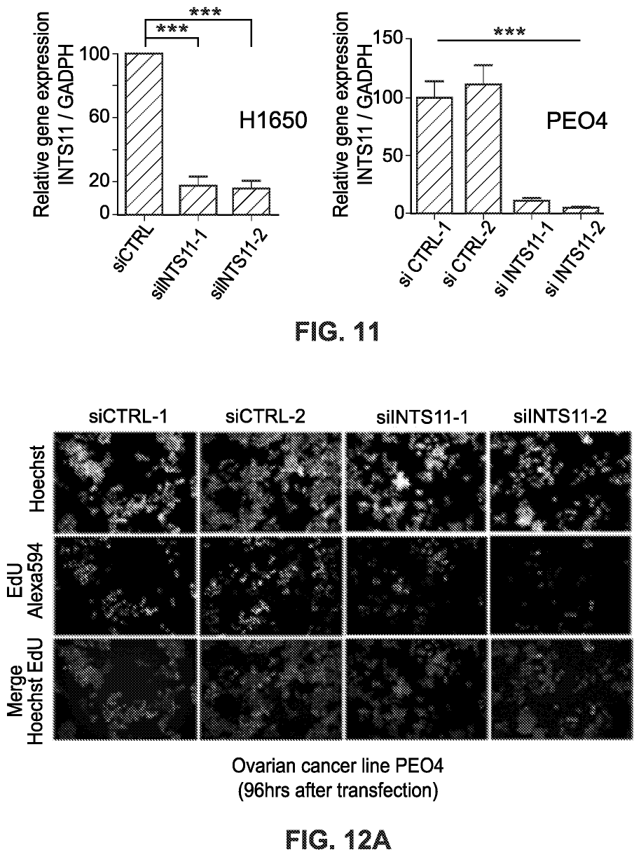

The NCI-H1650 lung cancer cell line containing heterozygous deletion in EGFR (delE746-A750) was treated with two different siRNAs to INTS11, or control non-targeting siRNAs, and cellular proliferation was measured using 5-ethynyl-2′-deoxyuridine (EdU), a sensitive and quantitative measure of cellular growth. Depletion of INTS11 specifically reduced proliferation of NCI-H1650 cells (FIG. 10A, 10B, and FIG. 11). Next three lung cancer cell lines, NCI-H1650, NCI-H1975 (EGFR T790M and L858R), and NCI-H2444 (KRAS G12V), were treated with EGFR inhibitors or phendione. Osimertinib was used to treat NCI-H1975, which contains mutations that make cells refractory to inhibition by Afatinib21. Two V600E BRAF-mutant melanoma cell lines, SK-MEL28 and A375, also were treated with increasing concentrations of phendione or Vemurafenib, a targeted inhibitor of V600E BRAF. SK-MEL28 also contains a homozygous EGFR mutation (P753S). Cell viability following treatment of cancer cells for 96 hours was compared with phendione, EGFR inhibitors (Osimertinib or Afatinib) or BRAF inhibitor (Vemurafenib). Phendione suppressed viability of all MAPK-driven cancer cell lines with IC₅₀s less than or equal to that of MAPK pathway inhibitors (FIG. 10C).

Next, the effectiveness of BRAF, MEK, ERK1/2 inhibitors or phendione was compared in blocking ERK1/2 activation and cellular growth suppression in either parental A375 cells or cells rendered resistant to BRAF inhibition. Interestingly, BRAF resistant cells were capable of activating ERK1/2 in the presence of MAPK pathway inhibitors, although to a lesser extent than in the absence of the inhibitors (FIG. 10D). Importantly, BRAF-resistant A375 cells were refractory to inhibition by the BRAF inhibitor and displayed decreased responsiveness to the MEK and ERK1/2 inhibitors (FIG. 10E). In contrast, resistant cells treated with phendione, which acts downstream of the kinase cascade, behaved identical to parental A375 (FIG. 10E). Without being bound by any particular theory, these results support the notion that Integrator functions downstream of the MAPK signaling network, and that inhibition of Integrator by phendione provides an effective means to treat BRAF mutant cells rendered resistant to BRAF inhibition.

### Phendione Inhibits Proliferation of C\Cisplatin-Resistant Ovarian Cancer

Interrogation of the cancer genome atlas (TCGA) indicated that multiple subunits of Integrator are amplified in ovarian cancers. Therefore, ovarian cancer cells were used to assess the effectiveness of targeting Integrator in cancers without activating mutations in the MAPK pathway. Phendione was compared with the conventional chemotherapeutic drug, cisplatin, a DNA crosslinking agent, which is frequently used in the treatment of the ovarian cancer. The effectiveness of INTS11 depletion in reducing the proliferation of PEO4 ovarian cancer cells was first measured (FIGS. 11, 12A, and 12B). Next, four ovarian cancer cell lines were treated with phendione or cisplatin. The two cell lines (A2780 and OVCAR10) with known resistance to cisplatin were less responsive to cisplatin treatment. Notably, all four ovarian cancer lines showed exquisite sensitivity in their cellular viability following phendione treatment with the two cisplatin insensitive cells (IC₅₀ of >8 μM for cisplatin) showing IC₅₀s of 0.3 and 1 μM for phendione (FIG. 12C). These results indicate that targeting Integrator is an effective strategy to treat MAPK-independent cancers that are insensitive to cisplatin and that phendione and cisplatin have distinct mechanisms of action.

### Integrator Inhibitors

Therefore, disclosed herein are compounds that act as inhibitors of INTS11 (a subunit of Integrator) catalytic activity, allowing them to suppress cancer activity without causing the resistance seen in traditional MAPK pathway inhibitors. These compounds can have a Formula (I), or can be a pharmaceutically acceptable salt thereof:

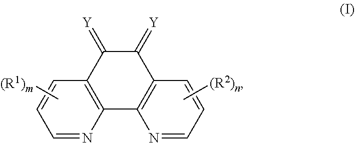

wherein
each Y independently is O or S;
each R¹ and R² is independently C₁₋₄alkyl or halo; and
each of m and n is independently 0, 1, 2, or 3.

In some embodiments, at least one Y is O. In various embodiments, each Y is O. I some cases, each Y is S. In various cases, each Y is S. In some exemplary embodiments, the compound of Formula (I) has a structure:

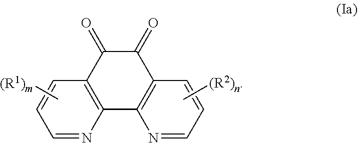

In various embodiments, R¹ and R² are each independently selected from the group consisting of methyl, ethyl, propyl, isopropyl, n-butyl, sec-butyl, tert-butyl, fluoro, chloro, bromo, and iodo. In some cases, each of R¹ and R² is C₁₋₄ alkyl. In various cases, each of R¹ and R² is halo. In some embodiments, one of R¹ and R² is C₁₋₄ alkyl and the other of R¹ and R² is halo. In some embodiments, at least one of R¹ and R² is methyl, ethyl, or propyl. In some cases, at least one of R¹ and R² is fluoro, chloro, or bromo.

In some cases, m is 0. In various cases, m is 1. In some embodiments, m is 2. In various embodiments, m is 3. In some cases, n is 0. In various cases, n is 1. In some embodiments, n is 2. In various embodiments, n is 3. In some embodiments, at least one of m and n is O. In various cases, each of m and n is 0. In some cases, at least one of m and n is 1 or 2 or 3. In some cases, only one of m and n is 1, 2, or 3.

In some exemplary embodiments, the compound of Formula (I) is 1,10-phenanthroline-5,6-dione:

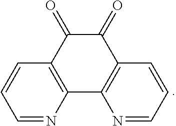 [SMILES: O=C1C(=O)c2cccnc2-c2ncccc21]

The compounds described herein, such as 1,10-phenanthroline-5,6-dione (“phendione”), have been found to inhibit the catalytic activity of Integrator and block MAPK-mediated transcriptional induction. These compounds (e.g., phendione) also have advantageously been found to display potent anti-proliferative activity toward a large number of human cancers, including those resistant to targeted therapies by EGFR- and BRAF-inhibitors.

### Methods

Also disclosed herein are methods of using the compounds of Formula (I) to inhibit the RNA polymerase II Integrator complex to attenuate growth signal response and to reduce the viability of cancer cells, including cancer cells that are resistant to MAPK pathway inhibitors.

Thus, in one aspect, the disclosure relates to a method of inhibiting Integrator in a cell. In this method, the cell is contacted with a compound of Formula (I), such as phendione, or a pharmaceutically acceptable salt thereof, in an amount effective to inhibit Integrator. In some embodiments, the compound of Formula (I) inhibits the Integrator subunit INTS11.

In another aspect, the disclosure relates to a method of suppressing aberrant MAPK signaling in a cell. In this method, the cell is contacted with a compound of Formula (I), such as phendione, or a pharmaceutically acceptable salt thereof, in an amount effective to suppress MAPK signaling.

In either of the above methods, the contacting can occur in vitro or in vivo. In some embodiments, the contacting occurs in vivo. In some embodiments, the cell can be resistant to MAPK pathway inhibitors (e.g., BRAF, MEK, or ERK inhibitors). In some cases, the contacting includes administering a compound of Formula (I), such as phendione, or a pharmaceutically acceptable salt thereof, to a patient in need thereof. In various cases, the patient suffers from a disease associated with deregulation of the mitogen-activated protein kinase pathway in a cell. In some embodiments, the disease can be cancer. For example, the cancer can include pancreatic, ovarian, prostate, breast, liver, uterine, bladder, lung, esophagus, diffuse large B-cell lymphoma, uveal melanoma, cholangiocarcinoma, stomach, sarcoma, testicular, malignant peripheral nerve sheath tumors, head and neck, mesothelioma, colorectal, cervical, and combinations thereof. In various cases, the cancer is a cisplatin-resistant cancer.

In yet another aspect, the disclosure relates to a method of treating a patient suffering from a disease associated with aberrant MAPK signaling. In this method, the patient is administered a therapeutically effective amount of a compound of Formula (I), such as phendione, or a pharmaceutically acceptable salt thereof. In some embodiments, the disease can be cancer, as previously described (e.g., pancreatic, ovarian, prostate, breast, liver, uterine, bladder, lung, esophagus, diffuse large B-cell lymphoma, uveal melanoma, cholangiocarcinoma, stomach, sarcoma, testicular, malignant peripheral nerve sheath tumors, head and neck, mesothelioma, colorectal, cervical, and combinations thereof), such as a cisplatin-resistant cancer.

Use of an inhibitor disclosed herein, such as a compound of Formula (I) (e.g., phendione), or a pharmaceutically acceptable salt thereof to treat a condition resulting from deregulation of the mitogen-activated protein kinase pathway complex in a patient, as well as use of the inhibitor in the preparation of a medicament for treating the condition, also are contemplated.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Pharmaceutical Formulations

Also provided herein are pharmaceutical formulations that include the inhibitors of the disclosure, and one or more pharmaceutically acceptable excipients.

The inhibitors of the disclosure can be administered to a subject or patient in a therapeutically effective amount. The inhibitors can be administered alone or as part of a pharmaceutically acceptable composition or formulation. In addition, the inhibitors can be administered all at once, as for example, by a bolus injection, multiple times, e.g. by a series of tablets, or delivered substantially uniformly over a period of time, as for example, using transdermal delivery. It is also noted that the dose of the compound can be varied over time.

The inhibitors disclosed herein and other pharmaceutically active compounds, if desired, can be administered to a subject or patient by any suitable route, e.g. orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g. eneteral or parenteral). All methods that can be used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The inhibitors of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### EXAMPLES

The following examples are provided for illustration and are not intended to limit the scope of the invention.

### RNA-Sequencing and Chromatin Immunoprecipitation Sequencing (“CHIP-Seq”)

RNA-sequencing and ChIP-sequencing were performed as described in Gardini, A. et al. Molecular Cell 56, 128-139, (2014) and Lai, F., Gardini, A., Zhang, A. & Shiekhattar, R. Nature 525, 399-403 (2015). In brief, the NEBNext Ultra RNA and the ChIP-Seq Library Prep Kits for Illumina (E7420, E6240, from New England Biolabs) were used to prepare the sequencing library. The sequencing was performed as a 75 bp single-end run using the NextSeq 500 High Output Kit provided by the Oncogenomics Core Facility at the Sylvester Comprehensive Cancer Center in the University of Miami Miller School of Medicine.

### RNA-Seq Analysis

RNA-seq data were aligned to human genome (hg19 version) using TopHat2, and differential expression analysis was performed using Cuffdiff 2.2.1 with default parameters. See Kim, D. et al. Genome Biol 14, R36, (2013); Trapnell, C. et al. Nature Biotechnology 28, 511-515, 1621 (2010). The differential expression was considered significant when the q-value <0.05, fold change >2 and FPKM >1 for protein coding genes and FPKM >0.5 for eRNAs. Heat maps were generated using SpotFire with Decision Site for Functional Genomics (SpotFire Inc., Somerville, Mass., USA).

Genome-Wide Identification of eRNA and Super-Enhancer RNA Loci

For eRNA identification, peak analysis was performed from HeLa H3K27ac ChIP-seq data after EGF stimulation (GSE68401) using HOMER (run in ‘histone’ mode). See Heinz, S. et al. Molecular Cell 38, 371 576-589 (2010). Chromatin-associated RNA-seq from HeLa cells (Vehicle and EGF) was used for transcriptome assembly with Cufflinks v2.2.1 with the following options: -N -u --library-type fr-firststrand -g (RefSeq GTF file provided as guide) -M (rRNA, tRNA and 7SK RNA mask file provided). See Trapnell, C. et al. Nature Biotechnology 28, 511-515, 1621 (2010). Transcriptome assemblies were generated for each of these samples separately and then Cuffmerge was used to combine all annotations. All spliced transcripts and any transcript that overlapped, or was in a window of, (±2 kb) of known RefSeq genes were removed. Next, BEDTools was used to retain all pairs of transcripts in a window of 500 nt that were head to head. See Quinlan, A. R. & Hall, I. M. Bioinformatics 26, 841-842, (2010). The pair of transcripts with TSS overlapping (±500 bp) with H3K27ac peaks was selected. This eRNA annotation was merged with the RefSeq and used for all subsequent RNA-Seq expression analyses. 75 EGF-induced eRNAs located within 300 kb from the nearest EGF-responsive protein-coding genes were selected for analysis. For super-enhancers, WT un-induced RNA-seq as “input” data and WT EGF induced RNA-seq as “ChIP-seq” data were used. In total, 3051 peaks were detected, and among them, 85 were called as super-enhancers (SEs). After manually removing protein-coding regions from the 85 SEs, 36 bona fide SEs were left. These 36 SEs and 464 traditional enhancers were combined to get the top 500 EGF induced enhancers. The enhancers are ranked by their Super Enhancer Score: normalized peak score based on the highest peak score and the total number of peaks (3051). Then, tag counts were quantified at those 500 non-redundant peaks from RNA-seq data of shINTS11, ERK inhibitor, and INTS11 inhibitor treated cells before and after EGF inductions. As the last step, the EGF induced tag was normalized to generate Super Enhancer Score in the same way as WT samples.

### ChIP-Seq Data Analysis and RNA Pol II Traveling Ratio (TR)

ChIP-seq data analysis was performed as previously described. See Lai, F., Gardini, A., Zhang, A. & Shiekhattar, R. Nature 525, 399-403, (2015). In brief, FASTQ data were processed with Trimmomatic to remove low-quality reads and then aligned to the human genome hg19 using bowtie2. See Bolger, A. M., Lohse, M. & Usadel, B. Bioinformatics 30, 2114-2120, (2014); Langmead, B. & Salzberg, S. L. Nat Methods 378 9, 357-359 (2012). The bigWiggle file was generated with samtools and RseQC and then uploaded to the UCSC Genome Browser. The average profile was generated with NGS Plot. See Shen, L., Shao, N., Liu, X. & Nestler, E. BMC Genomics 15, 284, (2014). RNAPII traveling ratio calculation were generated as described. See Rahl, P. B. et al. c-Myc regulates transcriptional pause release. Cell 141, 432-445, (2010). In brief, RNAPII ChIP-seq density at the TSS (−30 bp to +300 bp) was divided by the read density over the rest of the gene body, plus an additional 1 kb beyond the transcription end site (TES). The log₁₀(ratio) of genes (EGF, control and ERK inhibitor treatment) were calculated using all different isoforms available in the Hg19 RefSeq Annotation Table that were considered express (FPKM >1 in EGF treatment conditions) in our analysis.

### Antibodies

Antibodies used for CHIP and immunoblot include: INTS11 (A301-274A, Bethyl Laboratories, Inc., Montgomery, Tex.), RNAPII (sc-899, Santa Cruz Biotechnology, Paso Robles, Calif.), GAPDH (sc-25778, Santa Cruz), phospo-ERK1/2 (#9101, Cell Signaling technology), ERK1/2 (#9102, Cell Signaling technology), Phospho-p90RSK (Thr359) (#8753, Cell Signaling technology), and RSK1 (#9333, Cell Signaling technology). Flag M2-conjugated beads (Sigma, 2220) were used for immunoprecipitation.

### Cell Lines.

Melanoma cell lines A375 and SK-MEL28, lung cancer cell lines A549, NCI-H1650, NCI-H2444 and NCI-H1975, ovarian cancer cell lines PEO4, A2780 and OVCAR10 were purchased from ATCC and maintained under suggested conditions. In order to generate RAF inhibitor resistant cell line, A375 cell was cultured in the medium containing 1 μM vemurafenib for more than 3 months till the acquired resistance developed.

siRNA Transfections.

Gene silencing was achieved by transfection siRNAs (20 nM final concentration) in Optimem media (Invitrogen) using lipofectamine RNAiMax (Invitrogen 21cat#13778-100) according to the manufacturer's protocol. The siRNAs were purchased from Ambion (siINTS11#1 cat#s29894, siINTS11#2 cat#s29895, negative control siRNA, cat#AM4611) and Qiagen (Negative control siRNA cat#1022076).

### Proliferation and Apoptosis Assays.

Cell proliferation was tested by incorporation of EdU (5-ethynyl-2′-deoxyuridine) in NCI-H1650 and PEO4 cells transfected with siRNAs to down-regulate INTS11. EdU incorporation was tested also in Hela cells harboring doxycycline-inducible shRNA cassette. Four days after INTS11 knockdown, EdU incorporation was performed for 2 hours. The cells were then fixed, permeabilized and processed using Click-iT EdU Imaging Kit Alexa594 (Molecular probes cat#C10339). Nuclei were stained with Hoechst. EdU incorporation was visualized and the images were acquired with a fluorescence microscope. The quantification of EdU positive cells was done with automated counting using a Thermo Scientific imaging platform (Cellomics ArrayScan VTI HCS).

### Compounds

Afatinib (S1011), Erlotinib (S7786), Cisplatin (S1166), Osimertinib (S7297), Vemurafenib (S1267), PD0325901 (S1036), and SCH772984 (S7101) were purchased from Selleck Chemicals (Houston, Tex.) and resuspended in DMSO or water (cisplatin). Phendione (1,10-Phenanthroline-5,6-dione, 496383) was purchased from Sigma-Aldrich (St. Louis, Mo.) and resuspended in DMSO.

### Viability Assay

The cells were plated 5000 per well in 96-well black plates with a clear bottom and maintained under normal conditions over night before the drug treatment. The cell viability was measured 4 days after treatment using PrestoBlue (Invitrogen cat No A13261). GraphPad Prism software was used to generate dose response curves and calculate IC₅₀ values. Each experiment was repeated at least three times.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A method of inhibiting Integrator, comprising contacting the cell with a compound of Formula (I), or a pharmaceutically acceptable salt thereof, in an amount effective to inhibit Integrator: 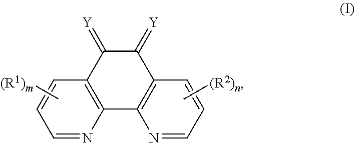
wherein
each Y independently is O or S;
each R¹ and R² is independently C₁₋₄alkyl or halo; and
each of m and n is independently 0, 1, 2, or 3.

2. A method of or suppressing MAPK signaling in a cell, comprising contacting the cell with a compound of Formula (I), or a pharmaceutically acceptable salt thereof in an amount effective to suppress MAPK cell signaling: 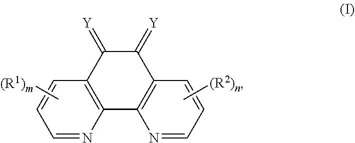
wherein
each Y independently is O or S;
each R¹ and R² is independently C₁₋₄alkyl or halo; and
each of m and n is independently 0, 1, 2, or 3.

3. The method of claim 1 or 2, wherein at least one Y is O.

4. The method of any one of claims 1 to 3, wherein each Y is O.

5. The method of any one of claims 1 to 4, wherein at least one of m and n is 0.

6. The method of any one of claims 1 to 5, wherein each of m and n is 0.

7. The method of any one of claims 1 to 5, wherein at least one of m and n is 1, 2, or 3.

8. The method of claim 7, wherein n is 1.

9. The method of claim 7 or 8, wherein m is 1.

10. The method of claim 7 or 9, wherein n is 2.

11. The method of claim 7, 8, or 10, wherein m is 2.

12. The method of any one of claims 7 to 11, wherein at least one of R¹ and R² is methyl, ethyl or propyl.

13. The method of any one of claims 7 to 12, wherein at least one of R¹ and R² is fluoro, chloro, or bromo.

14. The method of any one of claims 1 to 13, wherein the compound is as a pharmaceutically acceptable salt.

15. The method of any one of claims 1 and 3 to 14, wherein the compound inhibits the Integrator subunit INTS11.

16. The method of any one of claims 1 to 15, wherein the contacting occurs in vivo.

17. The method of any one of claims 1 to 16, wherein the contacting comprises administering to a patient in need thereof.

18. The method of claim 17, wherein the patient suffers from a disease associated with aberrant MAPK signaling in a cell.

19. The method of claim 18, wherein the disease is cancer.

20. The method of claim 19, wherein the cancer is selected from the group consisting of pancreatic, ovarian, prostate, breast, liver, uterine, bladder, lung, esophagus, diffuse large B-cell lymphoma, uveal melanoma, cholangiocarcinoma, stomach, sarcoma, testicular, malignant peripheral nerve sheath tumors, head and neck, mesothelioma, colorectal, cervical, and combinations thereof.
